(19) United States
(12) Patent Application Publication
(10) Pub. No.: US 2025/0082194 A1
(43) Pub. Date: Mar. 13, 2025

(54) SYNTHETIC LIGHT SOURCE GENERATION APPARATUS, PUPIL DIAMETER VARIATION INDUCING APPARATUS, METHODS, AND PROGRAMS THEREOF

(71) Applicant: NIPPON TELEGRAPH AND TELEPHONE CORPORATION, Tokyo (JP)

(72) Inventors: Yuta SUZUKI, Tokyo (JP); Shigeto FURUKAWA, Tokyo (JP)

(73) Assignee: NIPPON TELEGRAPH AND TELEPHONE CORPORATION, Tokyo (JP)

(21) Appl. No.: 18/580,546

(22) PCT Filed: Jul. 28, 2021
(86) PCT No.: PCT/JP2021/027896

§ 371 Date: Jan. 18, 2024

(51) Int. Cl. A61B 3/00; A61B 5/16; G16H 50/30

(52) U.S. Cl. CPC A61B 3/0008; A61B 5/163; A61B 5/165; G16H 50/30

(57) ABSTRACT

In a first time section, the output intensity of each basic light source of a plurality of basic light sources having different spectral distributions is adjusted to present a first synthetic source light with the plurality of basic light sources. In a second time section different from the first time section, the output intensity of each basic light source of the plurality of basic light sources is adjusted to present a second synthetic source light with the plurality of basic light sources. Here, the rate of change between the ipRGC activation amount of the first synthetic source light and the ipRGC activation amount of the second synthetic source light is higher than the rate of change between the value of the LMS chromaticity space of the first synthetic source light and the value of the LMS chromaticity space of the second synthetic source light.

### TECHNICAL FIELD

The present invention relates to a stealth light source that induces a change in pupil diameter.

### BACKGROUND ART

Human photoreceptor cells include, in addition to rod cells and cone cells, intrinsically photosensitive retinal ganglion cells (ipRGCs), and humans can perceive color and brightness through these cells. It is known that ipRGCs perceive brightness independently of color, and pupil contraction occurs when a stimulus with a high ipRGC activation amount is given to the eye (see Non Patent Literature 1, for example).

There also is a known method for estimating an attention target different from a visual stimulus, a change in the autonomic nervous system, or the like, using a change (such as pupil vibration, miosis/mydriasis, pupillary frequency tagging (PFT), or the like) induced in pupil diameter by the visual stimulus (see Non Patent Literature 2, for example).

### CITATION LIST

### Non Patent Literature

Non Patent Literature 1: S. Tsujimura and K. Ukai, “Contribution of human melanopsin retinal ganglion cells to steady-state pupil responses,” Proceedings of the Royal Society B: Biological Sciences, vol. 277, no. 1693, pp. 2485-2492, 2010.
Non Patent Literature 2: Naber, M., Alvarez, G. A., and Nakayama, K., “Tracking the allocation of attention using human pupillary oscillations,” [online], Dec. 10, 2013, Frontiers in Psychology, 4., [Searched on Jul. 15, 2021], Internet <http://doi.org/10.3389/fpsyg.2013.00919>

### SUMMARY OF INVENTION

### Technical Problem

However, a visual stimulus for inducing a change in pupil diameter normally involves a perceptually high-load operation, such as alternately displaying high- and low-luminance images on a monitor or repeatedly switching on/off an LED light source. Although it is also known that pupil contraction occurs when a stimulus with a high ipRGC activation amount is given to the eye, any technique using this for inducing pupil oscillation through a change in pupil diameter is not known yet.

The present invention has been made in view of such an aspect, and aims to provide a technology for inducing a change in pupil diameter with a perceptually low-load visual stimulus.

### Solution to Problem

In a first time section, the output intensity of each basic light source of a plurality of basic light sources having different spectral distributions is adjusted to present a first synthetic source light with the plurality of basic light sources. In a second time section different from the first time section, the output intensity of each basic light source of the plurality of basic light sources is adjusted to present a second synthetic source light with the plurality of basic light sources. Here, the rate of change between the ipRGC activation amount of the first synthetic source light and the ipRGC activation amount of the second synthetic source light is higher than the rate of change between the value of the LMS chromaticity space of the first synthetic source light and the value of the LMS chromaticity space of the second synthetic source light.

### Advantageous Effects of Invention

According to the present invention, ipRGC activation amounts are changed, while changes in the values of the LMS chromaticity spaces of synthetic source lights are reduced. By this technique, a pupil diameter change can be induced with a perceptually low-load visual stimulus.

### BRIEF DESCRIPTION OF DRAWINGS

FIG. 1 is a block diagram illustrating an example functional configuration of a pupil diameter change inducing system according to a first embodiment.

FIG. 2A is a block diagram illustrating an example functional configuration of a synthetic light source generation device according to an embodiment. FIG. 2B is a block diagram illustrating an example functional configuration of a pupil diameter change induction device according to an embodiment.

FIG. 3A is a block diagram illustrating an example functional configuration of a weight coefficient generation unit according to an embodiment. FIG. 3B is a flowchart for illustrating an example process to be performed by a weight coefficient generation unit according to an embodiment.

FIG. 4 is a graph showing an example of a spectral distribution (sensitivity distribution) L\hat{ }(λ) in the long-wavelength region (L region) based on a reaction of cone cells, a spectral distribution M\hat{ }(λ) in the middle-wavelength region (M region), a spectral distribution S\hat{ }(λ) in the short-wavelength region (S region), and a spectral distribution ipRGC\hat{ }(λ) of ipRGCs.

FIG. 5 is a graph showing an example of spectral distributions (radiance distributions) of a plurality of LEDs (basic light sources).

FIG. 6 is a graph for showing an example of a synthetic source light based on an LED 1 and an LED 2.

FIG. 7 is a block diagram illustrating an example hardware configuration of a synthetic light source generation device and a pupil diameter change induction device.

### DESCRIPTION OF EMBODIMENTS

The following is a description of embodiments of the present invention, with reference to the drawings.

### Principles

First, the principles are described. As described above, it has been known that pupil contraction occurs when a stimulus with a high ipRGC activation amount is given to the eye (see Non Patent Literature 1, for example). The present inventors have invented a technique for presenting, as a stealth stimulus, synthetic source light in which the ipRGC activation amount changes while changes in perceptual color are prevented, using the natural law (physiological law) that the size of the pupil diameter changes with the ipRGC activation amount. By this technique, a pupil diameter change can be induced with a perceptually low-load visual stimulus. This aspect is described below in detail.

### <Target Source Light>

First, a spectrum of source light that induces a change in pupil diameter is estimated. The estimated target source light will be hereinafter referred to as the “target source light”. The target source light may be of any kind, but a source light obtained by standardizing ordinary ambient light, such as the CIE standard light source D65, can be used. The values L, M, and S (target LMS chromaticity space values) of the LMS chromaticity space (also referred to as the “LMS color space”) of the target source light are expressed by Expressions (1) to (3) shown below, for example.

$$L = {\int }_{f 1}^{f 2} I ( λ ) * \hat{L} ( λ ) d λ \qquad ( 1 )$$

$$M = {\int }_{f 1}^{f 2} I ( λ ) * \hat{M} ( λ ) d λ \qquad ( 2 )$$

$$S = {\int }_{f 1}^{f 2} I ( λ ) * \hat{S} ( λ ) d λ \qquad ( 3 )$$

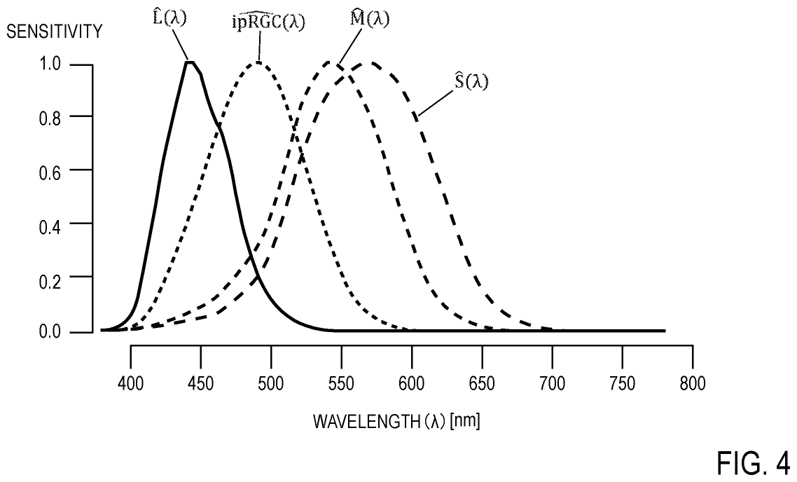

Here, λ represents wavelength [nm], and L\hat{ }(λ), M\hat{ }(λ), and S\hat{ }(λ) represent the spectral distributions (sensitivity distributions) in the long-wavelength region (L region), the middle-wavelength region (M region), and the short-wavelength region (S region), respectively. Note that the upper right suffix of “α\hat{ }” should be put directly above a (see Expression (1), for example), but are sometimes written as “α\hat{ }” due to the restrictions on notations. L\hat{ }(λ), M\hat{ }(λ), and S\hat{ }(λ) are set beforehand on the basis of the average human cone cell reaction. FIG. 4 shows an example of L\hat{ }(λ), M\hat{ }(λ), and S\hat{ }(λ). I(λ) represents the spectral distribution (radiance distribution) of the target source light. Further, f1 and f2 are positive real numbers indicating wavelengths, and satisfy f1<f2. Here, f1 and f2 may be any number as long as the region from f1 to f2 includes the visible light region. For example, f1 represents a lower limit wavelength (380 nm) in the visible light region, and f2 represents an upper limit wavelength (780 nm) in the visible light region. Further, “*” represents a multiplication operator.

Meanwhile, the activation amount ipRGC (ipRGC activation amount) of an ipRGC for the target source light is as shown below in Expression (4), for example.

$$ipRGC = {\int }_{f 1}^{f 2} I ( λ ) \hat{ipRGC} ( λ ) d λ \qquad ( 4 )$$

Here, ipRGC\hat{ }(λ) represents the spectral distribution (sensitivity distribution) of the ipRGC activation amount. Also, ipRGC\hat{ }(λ) is set beforehand on the basis of the average human ipRGC reaction. FIG. 4 shows an example of ipRGC\hat{ }(λ). In FIG. 4, the abscissa axis indicates wavelength (λ) [nm], and the ordinate axis indicates sensitivity.

### <Basic Light Sources>

Meanwhile, with N being an integer of 2 or greater, N (a plurality of) basic light sources U₁, . . . and U_{N} are set. The spectral distributions (radiance distributions) LED₁(λ), . . . , and LED_{N}(λ) of the basic source lights US₁, . . . , and US_{N} emitted from the basic light sources U₁, . . . , and U_{N} are different from one another, and, for example, the spectral distributions LED₁(λ), . . . , and LED_{N}(λ) have peak wavelengths different from one another. An example of a basic light source Uₙ (where n=1, . . . , N) is an LED light source. However, this does not limit the present invention, and any light source may be used as the basic light source Uₙ. The values of the LMS chromaticity space (the unit LMS chromaticity space values) of the nth basic source light USₙ among the plurality of basic source lights US₁, . . . , and US_{N} are represented by L_{LEDn}, M_{LEDn}, and S_{LEDn}. Note that n=1, . . . , N, and L_{LEDn}, M_{LEDn}, and S_{LEDn} are as shown below in Expressions (5) to (7), for example.

$${L}_{{LED}_{n}} = {\int }_{f 1}^{f 2} {LED}_{n} ( λ ) * \hat{L} ( λ ) d λ \qquad ( 5 )$$

$${M}_{{LED}_{n}} = {\int }_{f 1}^{f 2} LE {D}_{n} ( λ ) * \hat{M} ( λ ) d λ \qquad ( 6 )$$

$${S}_{{LED}_{n}} = {\int }_{f 1}^{f 2} {LED}_{n} ( λ ) * \hat{S} ( λ ) d λ \qquad ( 7 )$$

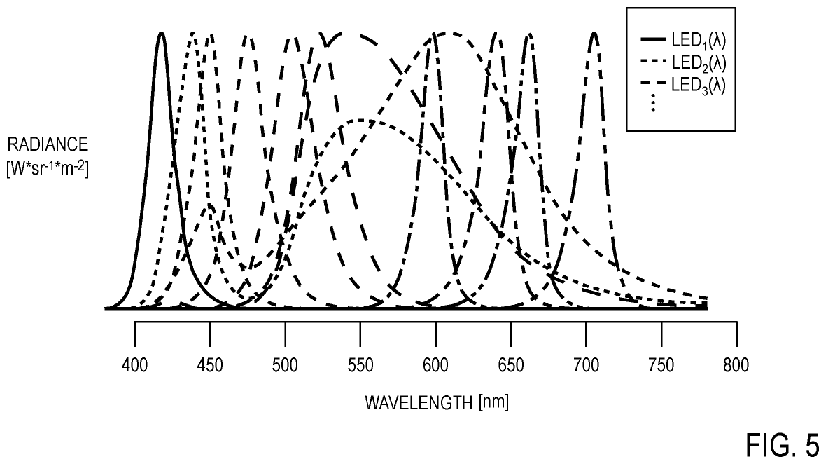

Here, the spectral distribution LEDₙ(λ) is obtained by measuring beforehand the basic source light USₙ emitted from the basic light source Uₙ. FIG. 5 shows twelve spectral distributions LED₁(λ), . . . , and LED₁₂(λ) (an example in which N=12). In FIG. 5, the abscissa axis indicates wavelength (λ) [nm], and the ordinate axis indicates radiance [W*sr⁻¹*m⁻²].

Further, the ipRGC activation amount ipRGC_{LEDn} (the basic ipRGC activation amount) for the basic source light USₙ is as shown below in Expression (8), for example.

$${ipRGC}_{{LED}_{n}} = {\int }_{f 1}^{f 2} {LED}_{n} ( λ ) * \hat{ipRGC} ( λ ) d λ \qquad ( 8 )$$

### <Generation of Synthetic Source Lights>

A case where a plurality of synthetic source lights s(i) having the same value in the LMS color space and different ipRGC activation amounts is generated with the use of at least some of the N basic light sources U₁, . . . , and U_{N} is now described. Note that I is an integer of 2 or greater, and i=1, . . . , I. Here, the spectral distribution Iₛ₍ᵢ₎(λ) of the synthetic source lights s(i) can be adjusted by selecting the basic light source to be used from among the N basic light sources U₁, . . . , and U_{N}. In a case where Q (Q being a positive integer of N or smaller) basic light sources U_{ϕ(1)}, . . . , and U_{ϕ(Q)} among the N basic light sources U₁, . . . , and U_{N} are used, there are _{N}C_{Q} combinations. The spectral distribution Iₛ₍ᵢ₎(λ) of the synthetic source lights s(i) can also be adjusted by controlling the mixing ratio of the spectral distributions LEDₙ(λ) of the respective basic light sources Uₙ. That is, the spectral distribution Iₛ₍ᵢ₎(λ) of the plurality of synthetic source lights is obtained by adjusting the mixing ratio using at least some basic light sources among the N basic light sources U₁, . . . , and U_{N}. Such a spectral distribution Iₛ₍ᵢ₎(λ) can be expressed by weighting and adding the spectral distributions LED₁(λ), . . . , and LED_{N}(λ) with weight coefficient combinations w₁(i), . . . , and w_{N}(i) as shown below in Expression (9).

$${I}_{s ( i )} ( λ ) = {w}_{1} ( i ) ⋆ L E {D}_{1} ( λ ) + … + {w}_{N} ( i ) ⋆ L E {D}_{N 1} ( λ ) \qquad ( 9 )$$

Note that w₁(i), . . . , and w_{N}(i) are real numbers of 0 or greater, and (w₁(i₁), . . . , w_{N}(i₁))≠(w₁(i₂), . . . , w_{N}(i₂)) at least for part of i₁, i₂∈{1, . . . , I} satisfying i₁≠i₂. The basic light source Uₙ corresponding to wₙ(i)=0 is not used (in other words, the spectral distribution LEDₙ(λ) corresponding to wₙ(i)=0 is not used). Here, the ith ipRGC activation amount that is set as appropriate for the target source light (the target ipRGC activation amount) is expressed as ipRGC(i). At least for part of i₁, i₂∈{1, . . . , I} satisfying i₁≠i₂, ipRGC(i₁)*ipRGC(i₂). In the above case, the weight coefficient combination w₁(i), . . . , and w_{N}(i) corresponding to the spectral distribution Iₛ₍ᵢ₎(λ) of the ith synthetic source light s(i) in which the values of the LMS color space are equal to the target LMS chromaticity space values L, M, and S, and the ipRGC activation amount is equal to the target ipRGC activation amount ipRGC(i) satisfies the following relationship.

$$Yw ( i ) = x ( i ) \qquad ( 10 )$$

Here, Y, w(i), and x(i) satisfy the following relationships.

$$Y = ( \begin{matrix}{L}_{{LED}_{1}} & … & {L}_{{LED}_{N}} \\ {M}_{{LED}_{1}} & … & {M}_{{LED}_{N}} \\ {S}_{{LED}_{1}} & … & {S}_{{LED}_{N}} \\ {ipRGC}_{{LED}_{1}} & … & {ipRGC}_{{LED}_{N}}\end{matrix} )$$

$$w ( i ) = ( \begin{matrix}{w}_{1} ( i ) \\ ⋮ \\ {w}_{N} ( i )\end{matrix} )$$

$$x ( i ) = ( \begin{matrix}L \\ M \\ S \\ ipRGC ( i )\end{matrix} )$$

The simultaneous equations specified by Expression (10) are solved, so that the weight coefficient combination w₁(i), . . . , and w_{N}(i) (w(i) representing the ith weight coefficient combination w₁(i), . . . , and w_{N}(i)) corresponding to the spectral distribution Iₛ₍ᵢ₎(λ) of the synthetic source light s(i) in which the values of the LMS color space are equal to the target LMS chromaticity space values L, M, and S, and the ipRGC activation amount is equal to the target ipRGC activation amount ipRGC(i) can be obtained. Weight coefficient combinations w₁(i), . . . , and w_{N}(i) different from one another are then obtained for target ipRGC activation amounts ipRGC(i) different from one another, so that spectral distributions Iₛ₍ᵢ₎(λ) of a plurality of synthetic source lights having the same value in the LMS color space and different ipRGC activation amounts can be generated (Expression (9)).

However, to implement the synthetic source lights s(i) of the spectral distributions Iₛ₍ᵢ₎(λ) calculated as described above, w₁(i), . . . , and w_{N}(i) need to be all 0 or greater, and each basic light source Uₙ needs to be capable of outputting light of a spectral distribution wₙ(i)*LEDₙ(λ). The former is a constraint based on the target ipRGC activation amount ipRGC(i) according to Expression (10), and the latter is a constraint based on the output performance (the maximum output and the minimum output) of each basic light source Uₙ. Therefore, in practice, it is necessary to select a combination in which w₁(i), . . . , and w_{N}(i) are all 0 or greater and light of the spectral distribution of wₙ(i)*LEDₙ(λ) can be output by each basic light source Uₙ, from among the weight coefficient combinations w₁(i), . . . , and w_{N}(i) obtained by solving the simultaneous equations specified by Expression (10).

Further, the values of the LMS color spaces of the plurality of spectral distributions Iₛ₍ᵢ₎(λ) obtained as described above are equal to each other and are L, M, and S, though L, M, and S are calculated with L\hat{ }(λ), M\hat{ }(λ), and S\hat{ }(λ) based on the average human cone cell reaction (Expressions (1), (2), and (3)). Because of this, the color sensation perceived from the synthetic source light s(i) having the spectral distribution Iₛ₍ᵢ₎(λ) does not always completely match the color sensation obtained from the target source light. Therefore, the obtained weight coefficient combination w₁(i), . . . , and w_{N}(i) may be corrected so that the color sensation perceived from the synthetic source light s(i) having the spectral distribution Iₛ₍ᵢ₎(λ) matches the color sensation from the target source light as much as possible. For example, the spectral distribution I(λ) of the target source light and the spectral distribution Iₛ₍ᵢ₎(λ) of the synthetic source light s(i) obtained according to Expression (9) may be converted into a predetermined color space, and the weight coefficient combination w₁(i), . . . , and w_{N}(i) may be corrected so that the colors of those distributions become similar to each other in the color space. For example, the spectral distribution I(λ) and the spectral distribution Iₛ₍ᵢ₎(λ) may be converted into CIExy chromaticity, and the weight coefficient combination w₁(i), . . . , and w_{N}(i) may be corrected so that the values thereof match or become close to each other in a CIExy chromaticity diagram. The conversion from the spectral distribution I(λ) to the CIExy chromaticity (x, y) can be performed according to Expressions (11) to (16) shown below, and the conversion from the spectral distribution Iₛ₍ᵢ₎(λ) to the CIExy chromaticity (xᵢ, yᵢ) can be performed according to Expressions (17) to (22) shown below.

$$X = k {\int }_{f 1}^{f 2} I ( λ ) * \hat{x} ( λ ) d λ \qquad ( 11 )$$

$$Y = k {\int }_{f 1}^{f 2} I ( λ ) * \hat{y} ( λ ) d λ \qquad ( 12 )$$

$$Z = k {\int }_{f 1}^{f 2} I ( λ ) * \hat{z} ( λ ) d λ \qquad ( 13 )$$

$$k = 1 / {\int }_{f 1}^{f 2} I ( λ ) * \hat{y} ( λ ) d λ \qquad ( 14 )$$

$$x = X / ( X + Y + Z ) \qquad ( 15 )$$

$$y = Y / ( X + Y + Z ) \qquad ( 16 )$$

$${X}_{i} = {k}_{i} {\int }_{f 1}^{f 2} {I}_{s ( i )} ( λ ) * \hat{x} ( λ ) d λ \qquad ( 17 )$$

$${Y}_{i} = {k}_{i} {\int }_{f 1}^{f 2} {I}_{s ( i )} ( λ ) * \hat{y} ( λ ) d λ \qquad ( 18 )$$

$${Z}_{i} = {k}_{i} {\int }_{f 1}^{f 2} {I}_{s ( i )} ( λ ) * \hat{z} ( λ ) d λ \qquad ( 19 )$$

$${k}_{i} = 1 / {\int }_{f 1}^{f 2} {I}_{s ( i )} ( λ ) * \hat{y} ( λ ) d λ \qquad ( 20 )$$

$${x}_{i} = {X}_{i} / ( {X}_{i} + {Y}_{i} + {Z}_{i} ) \qquad ( 21 )$$

$${y}_{i} = {Y}_{i} / ( {X}_{i} + {Y}_{i} + {Z}_{i} ) \qquad ( 22 )$$

Here, x\hat{ }(λ), y\hat{ }(λ), and z\hat{ }(λ) are CIE color matching functions indicating the respective spectral sensitivities of the tristimulus values of CIE.

Note that, in some cases, the corrected weight coefficient combination w₁(i), . . . , and w_{N}(i) does not match the weight coefficient combination w₁(i), . . . , and w_{N}(i) that satisfies the relationship shown in Expression (10), and is close to this combination. In this case, the values of the LMS color space of the synthetic source light having the spectral distribution Iₛ₍ᵢ₎(λ) obtained by substituting the corrected weight coefficient combination w₁(i), . . . , and w_{N}(i) into Expression (9) do not exactly match the target LMS chromaticity space values L, M, and S, but are close to these values L, M, and S. As a result, at least for part of i₁, i₂∈{1, . . . , I} satisfying i₁≠i₂, the values (Lₛ₍ᵢ₁₎, Mₛ₍ᵢ₁₎, and Sₛ₍ᵢ₁₎) of the LMS color space of the synthetic source light s(i₁) having the spectral distribution Iₛ₍ᵢ₁₎(λ) obtained by substituting w₁(i₁), . . . , and w_{N}(i₁) (the weight coefficient combination before or after correction) into Expression (9) do not match but are close to the values (Lₛ₍ᵢ₂₎, Mₛ₍ᵢ₂₎, and Sₛ₍ᵢ₂₎) of the LMS color space of the synthetic source light s(i₂) having the spectral distribution Iₛ₍ᵢ₂₎(λ) obtained by substituting w₁(i₂), . . . , and w_{N}(i₂) (the weight coefficient combination before or after correction) into Expression (9). Even in such a case, the rate of change between the ipRGC activation amount ipRGCₛ₍ᵢ₁₎ of the synthetic source light s(i₁) having the spectral distribution Iₛ₍ᵢ₁₎(λ) and the ipRGC activation amount ipRGCₛ₍ᵢ₂₎ of the synthetic source light s(i₂) having the spectral distribution Iₛ₍ᵢ₂₎(λ) is higher than the rate of change between the values (Lₛ₍ᵢ₁₎, Mₛ₍ᵢ₁₎, and Sₛ₍ᵢ₁₎) of the LMS chromaticity space of the synthetic source light s(i₁) and the values (Lₛ₍ᵢ₂₎, Mₛ₍ᵢ₂₎, and Sₛ₍ᵢ₂₎) of the LMS chromaticity space of the synthetic source light s(i₂). Note that ipRGCₛ₍ᵢ₁₎ is obtained from the right side of Expression (4) where I(λ)=Iₛ₍ᵢ₁₎(λ), and ipRGCₛ₍ᵢ₂₎ is obtained from the right side of Expression (4) where I(λ)=Iₛ₍ᵢ₂₎(λ). Lₛ₍ᵢ₁₎ is obtained from the right side of Expression (1) where I(λ)=Iₛ₍ᵢ₁₎(λ), and Lₛ₍ᵢ₂₎ is obtained from the right side of Expression (1) where I(λ)=Iₛ₍ᵢ₂₎(λ). Mₛ₍ᵢ₁₎ is obtained from the right side of Expression (2) where I(λ)=Iₛ₍ᵢ₁₎(λ), and Mₛ₍ᵢ₂₎ is obtained from the right side of Expression (2) where I(λ)=Iₛ₍ᵢ₂₎(λ). Sₛ₍ᵢ₁₎ is obtained from the right side of Expression (3) where I(λ)=Iₛ₍ᵢ₁₎(λ), and Sₛ₍ᵢ₂₎ is obtained from the right side of Expression (3) where I(λ)=Iₛ₍ᵢ₂₎(λ). Further, the above rate of change may be any index that is a scale-invariant index obtained by normalizing the absolute values of the amounts of change in the values of the ipRGCs or the LMS color spaces, such as relative errors, for example, between the synthetic source light s(i1) and the synthetic source light s(i2). Note that the subscripts “i1” and “i2” should be originally written as “i₁” and “i₂”, respectively, but may be written as “i₁” and “i₂”, respectively, due to the restrictions on notations.

As described above, the weight coefficient combinations w₁(i), . . . , and w_{N}(i) before or after correction are obtained for a plurality of i∈{1, . . . , I}, and the output intensity of each of the basic light sources U₁, . . . , and U_{N} is adjusted on the basis of these combinations (the output intensity of each basic light source Uₙ is adjusted in accordance with wₙ(i)), so that it is possible to generate a plurality of synthetic source lights s(i) (having the spectral distributions Iₛ₍ᵢ₎(λ) according to Expression (9)) in which the values of the LMS color spaces are equal or close to each other while the ipRGC activation amounts are different. That is, at least for part of i₁, i₂∈{1, . . . , I} satisfying i₁≠i₂, weight coefficient combinations w₁(i₁), . . . , and w_{N}(i₁) (the first weight coefficient combination) and w₁(i₂), . . . , and w_{N}(i₂) (the second weight coefficient combination) before or after correction are obtained as described above. The output intensity of each of the basic light sources U₁, . . . , and U_{N} is then adjusted in accordance with the weight coefficient combination w₁(i₁), . . . , and w_{N}(i₁) (the output intensity of each of the basic light sources Uₙ is adjusted in accordance with wₙ(i₁)), so that the synthetic source light s(i₁) (the second synthetic source light) can be obtained. Also, the output intensity of each of the basic light sources U₁, . . . , and U_{N} is adjusted in accordance with the weight coefficient combination w₁(i₂), . . . , and w_{N}(i₂) (the output intensity of each of the basic light sources Uₙ is adjusted in accordance with wₙ(i₂)), so that the synthetic source light s(i₂) (the second synthetic source light) can be obtained. The values of the LMS color spaces of the synthetic source light s(i₁) and the synthetic source light s(i₂) obtained in the above manner are equal or close to each other, while the ipRGC activation amounts are different from each other. In other words, the rate of change between the ipRGC activation amount of the synthetic source light s(i₁) and the ipRGC activation amount of the synthetic source light s(i₂) is higher than the rate of change between the values of the LMS chromaticity space of the synthetic source light s(i₁) and the values of the LMS chromaticity space of the synthetic source light s(i₂).

Note that adjusting the output intensity of a basic light source Uₙ in accordance with wₙ(i) means uniformly adjusting the output intensity of the entire visible light region of the basic light source Uₙ in accordance with wₙ(i). For example, adjusting the output intensity of a basic light source Uₙ in accordance with wₙ(i) means uniformly multiplying the output intensity of the entire visible light region of the basic light source Uₙ by β(wₙ(i)). Here, β(wₙ(i)) is a monotonically increasing function value of wₙ(i), and β(wₙ(i))=const*wₙ(i) for a positive real constant const, for example.

Meanwhile, the basic light source Uₙ corresponding to wₙ(i)=0 is not used, as mentioned above. Therefore, among the weight coefficient combinations w₁(i), . . . , and w_{N}(i), only the weight coefficient combinations satisfying wₙ(i)>0 are obtained, and the output intensity of each of the basic light sources U₁, . . . , and U_{N} may be adjusted on the basis of these combinations. In this case, the basic light sources Uₙ from which weight coefficients are not obtained are turned off (an unlit state).

Specifically, at least for i₁, i₂∈{1, . . . , I} satisfying i₁≠i₂, at least some elements of a weight coefficient combination close to w(i₁) or w(i₁) that satisfies Expression (10) are set as a weight coefficient combination w_{ϕ(1, 1)}(i₁), . . . , and w_{ϕ(1, N1)}(i₁) (the first weight coefficient combination), and at least some elements of a weight coefficient combination close to w(i₂) or w(i₂) that satisfies Expression (10) are set as a weight coefficient combination w_{ϕ(2, 1)}(i₂), . . . , and w_{ϕ(2, N2)}(i₂) (the second weight coefficient combination). Here, {ϕ(1, 1), . . . , ϕ(1, N1)}⊆{1, . . . , N}, and {ϕ(2, 1), . . . , ϕ(2, N2)}⊆{1, . . . , N}. In this case, the synthetic spectral distribution Iₛ₍ᵢ₁₎(λ) of the synthetic source light s(i₁) obtained by weighting and adding the spectral distributions LED_{ϕ(1, 1)}(λ), . . . , and LED_{ϕ(1, N1)}(λ) of a plurality of basic source lights US_{ϕ(1, 1)}, . . . , and US_{ϕ(1, N1)} with w_{ϕ(1, 1)}(i₁), . . . , and w_{ϕ(1, N1)}(i₁) can be expressed as shown below in Expression (23).

$${I}_{s ( i 1 )} ( λ ) = {w}_{ϕ ( 1 , 1 )} ( {i}_{1} ) ⋆ L E {D}_{ϕ ( 1 , 1 )} ( λ ) + … + {w}_{ϕ ( 1 , N 1 )} ( {i}_{1} ) ⋆ L E {D}_{ϕ ( 1 , N 1 )} ( λ ) \qquad ( 23 )$$

Also, the synthetic spectral distribution Iₛ₍ᵢ₂₎(λ) of the synthetic source light s(i₂) obtained by weighting and adding the spectral distributions LED_{ϕ(2, 1)}(λ), . . . , and LED_{ϕ(2, N2)}(λ) of a plurality of basic source lights US_{ϕ(2, 1)}, . . . , and US_{ϕ(2, N2)} with w_{ϕ(2, 1)}(i₂), . . . , and w_{ϕ(2, N2)}(i₂) can be expressed as shown below in Expression (24).

Iₛ₍ᵢ₂₎(λ)=w_{ϕ(2,1)}(i₂)*LED_{ϕ(2,1)}(λ)+ . . . +w_{ϕ(2,N2)}(i₂)*LED_{ϕ(2,N2)}(λ) (24)

### <Presentation of Synthetic Source Lights>

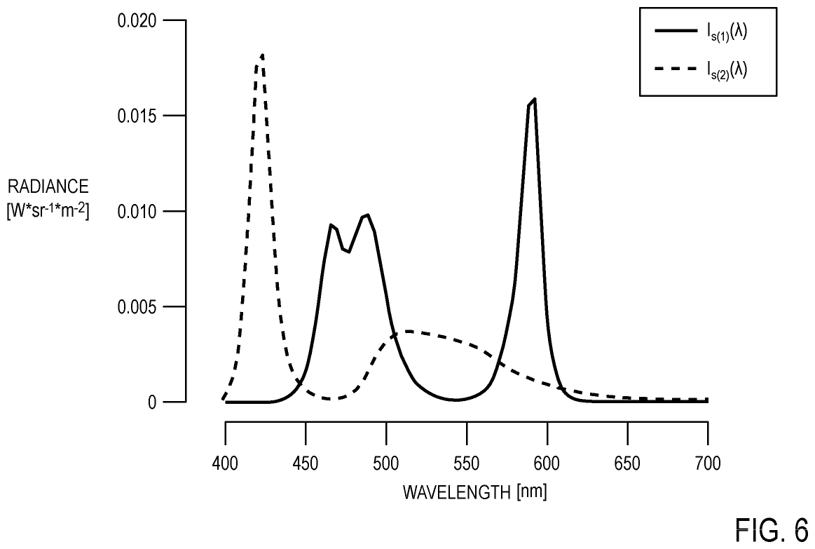

The plurality of synthetic source lights s(i) generated as described above are presented (output, emitted, or displayed) while being switched in accordance with time. For example, at least for i₁, i₂∈{1, . . . , I} satisfying i₁≠i₂, in a time section T(i₁), the output intensity of each of the basic light sources U_{ϕ(1, 1)}, . . . , and U_{ϕ(1, N1)} emitting the basic source lights US_{ϕ(1, 1)}, . . . , and US_{ϕ(1, N1)} is adjusted in accordance with the weight coefficient combination w_{ϕ(1, 1)}(i₁), . . . , and w_{ϕ(1, N1)}(i₁), and the synthetic source light s(i₁) is presented by the basic light sources U_{ϕ(1, 1)}, . . . , and U_{ϕ(1, N1)}. Further, in a time section T(i₂) different from the time section T(i₁), the output intensity of each of the basic light sources U_{ϕ(2, 1)}, . . . , and U_{ϕ(2, N2)} is adjusted in accordance with the weight coefficient combination w_{ϕ(2, 1)}(i₂), . . . , and w_{ϕ(2, N2)}(i₂), and the synthetic source light s(i₂) is presented by the plurality of basic light sources U_{ϕ(2, 1)}, . . . , and U_{ϕ(2, N2)}. FIG. 6 shows an example of the spectral distribution Iₛ₍ᵢ₁₎(λ) of the synthetic source light s(i₁) and the spectral distribution Iₛ₍ᵢ₂₎(λ) of the synthetic source light s(i₂). In FIG. 6, the abscissa axis indicates wavelength (λ) [nm], and the ordinate axis indicates radiance [W*sr⁻¹*m⁻²]. The values of the LMS color spaces of the synthetic source light s(i₁) and the spectral distribution Iₛ₍ᵢ₂₎(λ) obtained in the above manner are equal or close to each other, while the ipRGC activation amounts are different from each other.

For example, the plurality of synthetic source lights s(i) is repeatedly presented while being switched in accordance with time. The plurality of synthetic source lights s(i) may be periodically switched and presented, or may be aperiodically switched and presented. For example, at least for i₁, i₂∈{1, . . . , I} satisfying i₁≠i₂, presentation of the synthetic source light s(i₁) and presentation of the synthetic source light s(i₂) are repeated.

The ipRGC activation amounts of the plurality of synthetic source lights s(i) to be presented while being switched are different from each other, which induces a change in pupil diameter of a person. Furthermore, the values of the LMS chromaticity spaces of the plurality of synthetic source lights s(i) to be presented are identical or close to each other, and these values do not greatly change. Thus, color changes to be perceived by a person are reduced. As a result, it is possible to induce a change in pupil diameter while reducing the perceptual load.

### First Embodiment

Next, a first embodiment is described with reference to the drawings.

### Overall Configuration

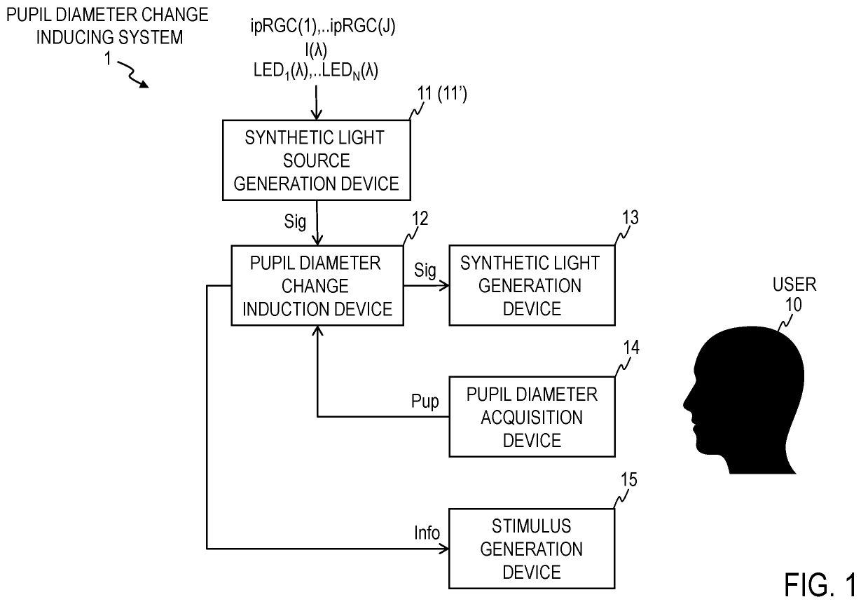

As illustrated in FIG. 1, a pupil diameter change inducing system 1 of this embodiment includes a synthetic light source generation device 11, a pupil diameter change induction device 12, a synthetic light generation device 13, a pupil diameter acquisition device 14, and a stimulus generation device 15.

### <Synthetic Light Source Generation Device 11>

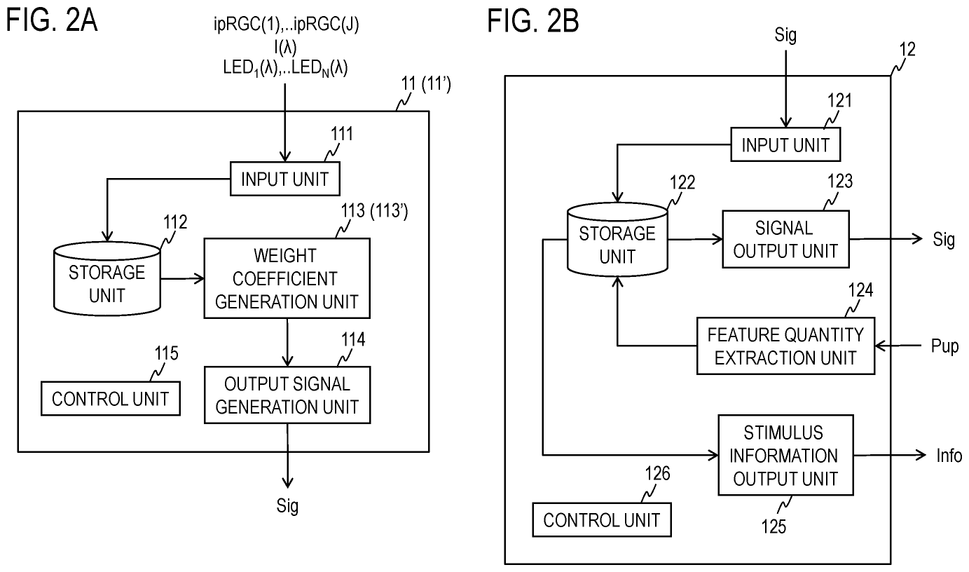

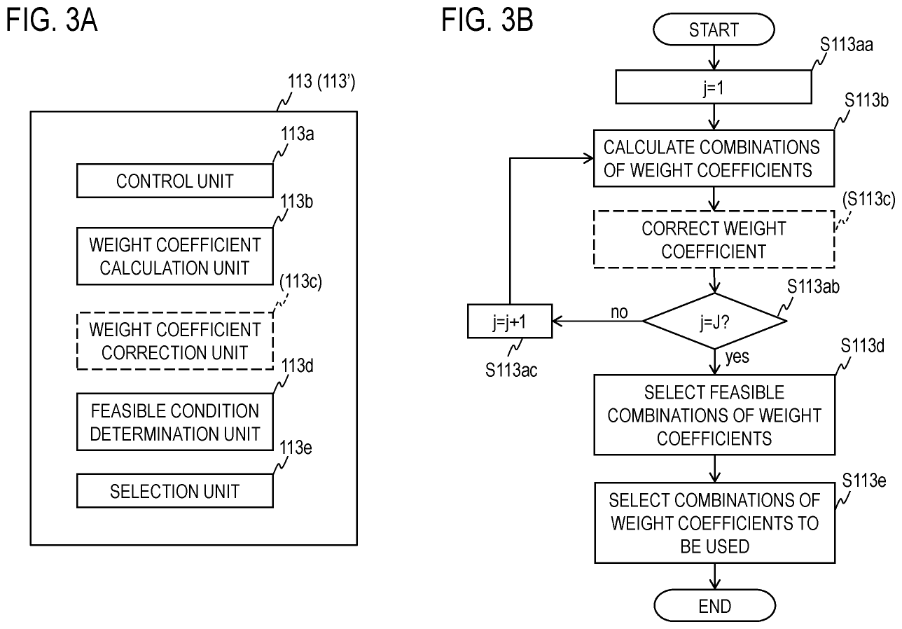

As illustrated in FIG. 2A, the synthetic light source generation device 11 of this embodiment includes an input unit 111, a storage unit 112, a weight coefficient generation unit 113, an output signal generation unit 114, and a control unit 115. As illustrated in FIG. 3A, the weight coefficient generation unit 113 includes a control unit 113a, a weight coefficient calculation unit 113b, a feasible condition determination unit 113d, and a selection unit 113e. Although not mentioned in the description below, data input to the synthetic light source generation device 11 and data obtained by the respective processing units are stored into the storage unit 112 piece by piece, are read out as necessary, and are used in the respective processes. The synthetic light source generation device 11 also performs each process, under the control of the control unit 115.

### <Pupil Diameter Change Induction Device 12>

As illustrated in FIG. 2B, the pupil diameter change induction device 12 includes an input unit 121, a storage unit 122, a signal output unit 123, a feature quantity extraction unit 124, a stimulus information output unit 125, and a control unit 126. Although not mentioned in the description below, data input to the pupil diameter change induction device 12 and data obtained by the respective processing units are stored into the storage unit 122 piece by piece, are read out as necessary, and are used in the respective processes. The pupil diameter change induction device 12 also performs each process, under the control of the control unit 126.

### <Synthetic Light Generation Device 13>

The synthetic light generation device 13 of this embodiment is a device that presents a synthetic source light s(i) for inducing a change in pupil diameter of a user 10. The synthetic light generation device 13 of this embodiment includes N (a plurality of) basic light sources U₁, . . . , and U_{N}. As described above, an example of a basic light source Uₙ (where n=1, . . . , N) is an LED light source. Output signals Sig=(Sig₁, . . . , Sig_{N}) that will be described later are input to the synthetic light generation device 13, and output intensities of the basic light sources U₁, . . . , and U_{N} are controlled on the basis of Sig₁, . . . , and Sig_{N}, respectively. Under this control, the basic light sources U₁, . . . , and U_{N} present the synthetic source lights s(i).

### <Pupil Diameter Acquisition Device 14>

The pupil diameter acquisition device 14 of this embodiment is a device that measures a pupil diameter of the user 10. For example, the pupil diameter acquisition device 14 includes a camera that captures movement of an eye of the user 10, and a device that acquires and outputs a pupil diameter Pub of the user 10 from a video image captured by the camera. An example of the pupil diameter acquisition device 14 is a commercially available eye tracker or the like.

### <Stimulus Generation Device 15>

The stimulus generation device 15 of this embodiment is a device for presenting (outputting) another sensory stimulus independent of the induction of a change in pupil diameter to the user 10. This sensory stimulus is a stimulus for a task (task) to be dealt with by the user 10, and may be a visual stimulus, an auditory stimulus, a tactile stimulus, an olfactory stimulus, or a taste stimulus. Examples of the stimulus generation device 15 include a monitor, a speaker, and a vibrator.

### <Synthetic Light Source Generation Process>

Next, a synthetic light source generation process to be performed by the synthetic light source generation device 11 of this embodiment is described.

Target ipRGC activation amounts ipRGC(1), . . . , and ipRGC(J) that are set as appropriate, a spectral distribution I(λ) of the target source light that is set as appropriate, and spectral distributions LED₁(λ), . . . , and LED_{N}(λ) of the basic source lights US₁, . . . , and US_{N} of the synthetic light generation device 13 measured beforehand are input to the input unit 111 of the synthetic light source generation device 11. Here, J is an integer of 2 or greater, j=1, . . . , J, and ipRGC(j₁)≠ipRGC(j₂) is satisfied at least for part of j₁, j₂∈{1, . . . , J}. The input ipRGC activation amounts ipRGC(1), . . . , and ipRGC(J), the spectral distribution I(λ), and the spectral distributions LED₁(λ), . . . , and LED_{N}(λ) are stored into the storage unit 112 (step S111).

The weight coefficient generation unit 113 reads the spectral distribution I(λ) and the spectral distributions LED₁(λ), . . . , and LED_{N}(λ) from the storage unit 112, and obtains the basic ipRGC activation amount ipRGC_{LEDn} for unit LMS chromaticity space values L_{LEDn}, M_{LEDn}, and S_{LEDn} (n=1, . . . , N) with respect to target LMS chromaticity space values L, M, and S (n=1, . . . , N), according to the above Expressions (1) to (3), and (5) to (8). Further, the weight coefficient generation unit 113 obtains weight coefficient combinations w₁(i), . . . , and w_{N}(i) (where i=1, . . . , I) (at least a first weight coefficient combination w₁(i₁), . . . , and w_{N}(i₁) and a second weight coefficient combination w₁(i₂), . . . , and w_{N}(i₂) for the spectral distributions of a plurality of basic source lights) as described above, using the basic ipRGC activation amount ipRGC_{LEDn} for the unit LMS chromaticity space values L_{LEDn}, M_{LEDn}, and S_{LEDn} (n=1, . . . , N) with respect to the target LMS chromaticity space values L, M, and S (n=1, . . . , N), and the target ipRGC activation amounts ipRGC(1), . . . , and ipRGC(J) read from the storage unit 112. This process will be described later in detail (step S113).

The weight coefficient combinations w₁(i), . . . , and w_{N}(i) are input to the output signal generation unit 114. Using the weight coefficient combinations w₁(i), . . . , and w_{N}(i), the output signal generation unit 114 obtains and outputs output signals Sig=(Sig₁, . . . , Sig_{N}). The output signals Sig=(Sig₁, . . . , Sig_{N}) include information (Sig₁(i), . . . , and Sig_{N}(i)) for adjusting the output intensity of each of the basic light sources U₁, . . . , and U_{N} in accordance with the weight coefficient combination w₁(i), . . . , and w_{N}(i) and presenting the synthetic source lights s(i) with the basic light sources U₁, . . . and U_{N} in each time section T(i). Here, Sig₁=(Sig₁(1), . . . , Sig₁(I)), . . . , Sig_{N}=(Sig_{N}(1), . . . , Sig_{N}(I)). However, for i₁, i₂∈{1, . . . , I} satisfying i₁≠i₂, the time interval T(i₁) and the time interval T(i₂) are different from each other. That is, the output signals Sig include at least (Sig₁(i₁), . . . , and Sig_{N}(i₁)) (first information) and (Sig₁(i₂), and Sig_{N}(i₂)) (second information). (Sig₁(i₁), . . . , and Sig_{N}(i₁)) is information for adjusting the output intensity of each of the basic light sources U_{ϕ(1, 1)}, . . . , and U_{ϕ(1, N1)} in accordance with the weight coefficient combination w_{ϕ(1, 1)}(i₁), . . . , and w_{ϕ(1, N1)}(i₁) and presenting the synthetic source light s(i₁) (Expression (23)) with the basic light sources U_{ϕ(1, 1)}, and U_{ϕ(1, N1)} in the time section T(i₁). (Sig₁(i₂), . . . , and Sig_{N}(i₂)) is information for adjusting the output intensity of each of the basic light sources U_{ϕ(2, 1)}, and U_{ϕ(2, N2)} in accordance with the weight coefficient combination w_{ϕ(2, 1)}(i₂), . . . , and w_{ϕ(2, N2)}(i₂) and presenting the synthetic source light s(i₂) (Expression (24)) with the basic light sources U_{ϕ(2, 1)}, . . . , and U_{ϕ(2, N2)} in a time section T(i₂) different from the time section T(i₁). An example of Sigₙ(i) is information for uniformly adjusting the output intensity of the entire visible light region of the basic light source Uₙ in accordance with wₙ(i), and indicates β(wₙ(i)), for example, for uniformly multiplying the output intensity of the entire visible light region of the basic light source Uₙ by β(wₙ(i)) (step S114).

### [Details of Step S113]

Referring now to FIGS. 3A and 3B, an example of step S113 is described in detail.

First, the control unit 113a initializes j to 1 (step S113aa). Next, the weight coefficient calculation unit 113b obtains weight coefficient combinations w₁(j), . . . , and w_{N}(j) by solving simultaneous equations specified by Expression (10) where j=i, and outputs the weight coefficient combinations (step S113b). Next, the control unit 113a determines whether j=J (step S113ab). If j=J is not satisfied, the control unit 113a sets j+1 as the new j, and returns the process to step S113b (step S113ac). On the other hand, if j=J is satisfied, the feasible condition determination unit 113d selects, from among the obtained weight coefficient combinations w₁(j), . . . , and w_{N}(j), combinations in which w₁(j), . . . , and w_{N}(j) (j=1, . . . , J) are all 0 or greater, and each basic light source Uₙ can output light of the spectral distribution of wₙ(j)*LEDₙ(λ). The feasible condition determination unit 113d outputs the selected weight coefficient combinations as w₁(j′), . . . , and w_{N}(j′) (j′=1, . . . , J′, and j′ is an integer of 2 or greater) (step S113d). The selection unit 113e outputs the weight coefficient combinations to be used as w₁(i), . . . , and w_{N}(i) (i=1, . . . , I), from the weight coefficient combinations w₁(j′), . . . , and w_{N}(j′) (j′=1, . . . , J′). All of w₁(j′), . . . , and w_{N}(j′) (j′=1, . . . , J′) may be w₁(i), . . . , and w_{N}(i) (i=1, . . . , I), or only some of w₁(j′), . . . , and w_{N}(j′) (j′=1, . . . , J′) may be w₁(i), . . . , w_{N}(i) (i=1, . . . , I). The latter selection criteria are not limited, but combinations of I coefficients w₁(i), . . . , and w_{N}(i) (i=1, . . . , I) may be selected by arranging the coefficients in descending order or ascending order of the difference in ipRGC activation amount, for example. Further, combinations of coefficients w₁(i), . . . , and w_{N}(i) are output at least for two types of i.

### <Pupil Diameter Change Induction Process>

The output signals Sig=(Sig₁, Sig_{N}) output from the synthetic light source generation device 11 are input to the input unit 121 of the pupil diameter change induction device 12 (FIG. 2B), and are stored into the storage unit 122 (step S121).

The signal output unit 123 outputs the output signals Sig read from the storage unit 122 to the synthetic light generation device 13. The synthetic light generation device 13 adjusts the output intensity of each of the basic light sources U₁, and U_{N} in accordance with the weight coefficient combinations w₁(i), . . . , w_{N}(i) in each time section T(i), and presents the synthetic source lights s(i) with the basic light sources U₁, . . . , and U_{N}. As a result, the synthetic source lights s(i) are switched in each time section T(i) where i=1, . . . , N, and are presented to the user 10. For example, for i₁, i₂∈{1, . . . , I} satisfying i₁≠i₂, the synthetic light generation device 13 presents the synthetic source light s(i₁) with the basic light sources U_{ϕ(1, 1)}, and U_{ϕ(1, N1)} in the time section T(i₁), and presents the synthetic source light s(i₂) with the basic light sources U_{ϕ(2, 1)}, . . . , and U_{ϕ(2, N2)} in the time section T(i₂) different from the time section T(i₁). For example, each synthetic source light s(i) is periodically or aperiodically presented in a repetitive manner while being switched. For example, the synthetic source light s(i₁) and the synthetic source light s(i₂) are alternately and repeatedly presented periodically or aperiodically. In a case where each synthetic source light s(i) is periodically and repeatedly presented, the repetitive frequency is referred to as the “blinking frequency” (step S123).

While the synthetic source light s(i) is presented, the stimulus information output unit 125 outputs information Info for presenting a sensory stimulus to the stimulus generation device 15. The stimulus generation device 15 presents visual stimuli such as a video image or a sound to the user 10, on the basis of the information Info. The user 10 is instructed to execute a task regarding the presented visual stimuli. For example, the user 10 is instructed to execute a task of directing attention to one of the presented visual stimuli (step S125).

The pupil diameter acquisition device 14 acquires and outputs a pupil diameter Pub of the user 10, who has executed the task regarding the presented visual stimuli and has a change induced in pupil diameter by the synthetic source lights s(i). The pupil diameter Pub is input to the feature quantity extraction unit 124 of the pupil diameter change induction device 12. The feature quantity extraction unit 124 extracts the feature quantity of the change in pupil diameter from time-series data of the pupil diameter Pub obtained by the pupil diameter acquisition device 14, and outputs the extracted feature quantity. The feature quantity of the change in pupil diameter may be of any kind. For example, the feature quantity extraction unit 124 extracts the feature quantity through the following procedures.

I. The feature quantity extraction unit 124 performs preprocessing on the time-series data of the pupil diameter Pub obtained by the pupil diameter acquisition device 14, and obtains time-series data of the pupil diameter Pub′ after the preprocessing. For example, the feature quantity extraction unit 124 interpolates a portion missing from the time-series data of the pupil diameter Pub due to blinking of the user 10 or the like, using linear interpolation, quadratic spline interpolation, or the like. Further, in a case where each synthetic source light s(i) is periodically and repeatedly presented, a low-pass filter corresponding to the blinking frequency (a low-pass filter that passes frequencies equal to or lower than the blinking frequency, for example) may be applied to the time-series data of the pupil diameter Pub subjected to the interpolation, to reduce noise.

II. The feature quantity extraction unit 124 subtracts the average value of the pupil diameter Pub′ prior to the execution of the task from the time-series data of the pupil diameter Pub′ during the execution of the task, and performs normalization with the z value, to obtain time-series data of the pupil diameter change VPub during the execution of the task.

III. The feature quantity extraction unit 124 performs Fourier transform on the time-series data of the pupil diameter change VPub, to obtain a frequency distribution FPub of the pupil diameter change. Further, the feature quantity extraction unit 124 sets the peak of the power of the frequency distribution FPub of the pupil diameter change corresponding to the blinking frequency as the feature quantity. Note that the peak of the power of the frequency distribution FPub of the pupil diameter change corresponding to the blinking frequency means the peak of the power of the blinking frequency or the power in the neighborhood of the blinking frequency in the peak of the power of the frequency distribution FPub of the pupil diameter change.

In addition to the above peak of the power of the frequency distribution FPub of the pupil diameter change, other information may be included in the feature quantity. For example, the feature quantity may include information that captures characteristics such as the peak of a doubled frequency of the peak of the power of the frequency distribution FPub of the pupil diameter change corresponding to the blinking frequency, the degree of synchronization of a phase change in the ipRGC activation amount of the synthetic source light s(i) and a phase change in the pupil diameter change VPub, or a cross-correlation between an aperiodic visual stimulus such as a video image or a sound presented from the stimulus generation device 15 and the pupil diameter change VPub (step S124).

### Modification 1 of the First Embodiment

In the description below, differences from the first embodiment are mainly explained, and the components already described are denoted by the same reference numerals so that explanation thereof is simplified. As described above, the weight coefficient combinations w₁(i), . . . , and w_{N}(i) may be corrected. In this case, the synthetic light source generation device 11 is replaced with a synthetic light source generation device 11′ (FIG. 1, FIG. 2A, FIG. 3A). The synthetic light source generation device 11′ has the weight coefficient generation unit 113 of the synthetic light source generation device 11 replaced with a weight coefficient generation unit 113′, and the weight coefficient generation unit 113′ is formed by further adding a weight coefficient correction unit 113c to the weight coefficient generation unit 113.

As illustrated in FIG. 3B, after performing the processes in steps S113aa and S113b described above, the weight coefficient generation unit 113′ converts the weight coefficient combinations w₁(j), . . . , and w_{N}(j) into a predetermined color space (CIExy chromaticity, for example), corrects the combinations w₁(j), . . . , and w_{N}(j) so that their values match or become close to each other in the color space, and outputs the corrected combinations w₁(j), . . . , and w_{N}(j). After that, the processes in steps S113ab, S113ac, S113d, and S113e are performed for the corrected combinations w₁(j), . . . , and w_{N}(j). The other aspects are the same as those of the first embodiment.

### Modification 2 of the First Embodiment

Y and x(i) may include other elements. For example, Y and x(i) shown below may be used.

$$Y = ( \begin{matrix}{L}_{{LED}_{1}} & … & {L}_{{LED}_{N}} \\ {M}_{{LED}_{1}} & … & {M}_{{LED}_{N}} \\ {S}_{{LED}_{1}} & … & {S}_{{LED}_{N}} \\ {ipRGC}_{{LED}_{1}} & … & {ipRGC}_{{LED}_{N}} \\ {y}_{51} & … & {y}_{5 N} \\ ⋮ & ⋱ & ⋮ \\ {y}_{p 1} & … & {y}_{pN}\end{matrix} )$$

$$x ( i ) = ( \begin{matrix}L \\ M \\ S \\ ipRGC ( i ) \\ {x}_{5} \\ ⋮ \\ {x}_{p}\end{matrix} )$$

Here, p is an integer of 5 or greater, and y₅₁, . . . y_{5N}, . . . , yₚ₁, . . . y_{pN}, and x₅, yₚ are appropriate values. Examples of y₅₁, . . . y_{5N}, . . . , yₚ₁, . . . y_{pN} include the values of the LMS color space and the ipRGC activation amounts of second basic light sources other than the basic light sources described above, and examples of x₅, . . . yₚ include the values of the LMS chromaticity space and the ipRGC activation amount of a second target source light other than the target source light described above.

### Second Embodiment

This embodiment is a further modification of the first embodiment and Modifications 1 and 2 thereof. For example, the synthetic source light s(i) described in the first embodiment may be used as a stealth stimulus for monitoring a stress state appearing in the autonomic nervous system of the user 10 when the user is driving a car or receiving a visual stimulus (a video image or the like). For example, in a state where the stress (the index of fatigue or recovery from fatigue) is high, it is expected that the pupil diameter change (light reflection) with respect to the synthetic source light s(i) becomes greater. Therefore, the signal output unit 123 of the first embodiment may present synthetic source lights s(i) to the user 10 while switching the synthetic source lights s(i), and the feature quantity extraction unit 124 may extract a feature quantity of the pupil diameter change from time-series data of the pupil diameter Pub of the user 10, and monitor the stress state appearing in the autonomic nervous system of the user 10 with the feature quantity.

### Summary

As described above, in each embodiment and its modifications, the ipRGC activation amounts of a plurality of synthetic source lights s(i) to be presented while being switched are different from each other, which induces a change in pupil diameter of a person. Furthermore, the values of the LMS chromaticity spaces of the plurality of synthetic source lights s(i) to be presented are identical or close to each other, and these values do not greatly change. Thus, color changes to be perceived by a person are reduced. As a result, it is possible to induce a change in pupil diameter while reducing the perceptual load.

Further, since the values of the LMS chromaticity space of the synthetic source lights s(i) do not change greatly, the synthetic source lights s(i) serve as a stealth stimulus that induces a change in pupil diameter. That is, with the synthetic source lights s(i) serving as a stealth stimulus, the user 10 can concentrate on sensory stimuli (other sensory stimulus independent of the induction of the change in pupil diameter) presented from the stimulus generation device 15. Thus, it is possible to reduce the task to be dealt with by the user 10 and the noise in the autonomic nervous system of the user 10, and, as a result, more robust data can be acquired.

### [Hardware Configuration]

The synthetic light source generation device 11 or 11′, and the pupil diameter change induction devices 12 in each embodiment are devices formed by a general-purpose or dedicated computer executing a predetermined program, the computer including a processor (hardware processor) such as a central processing unit (CPU), and a memory such as a random-access memory (RAM) and a read-only memory (ROM), for example. That is, the synthetic light source generation device 11 or 11′, and the pupil diameter change induction devices 12 in the respective embodiments each have processing circuitry configured to implement each of the components included therein, for example. The computer may include one processor and one memory, or may include a plurality of processors and a plurality of memories. The program may be installed into the computer, or may be recorded in a ROM or the like in advance. Also, some or all of the processing units may be formed with an electronic circuit that independently implements the processing functions, rather than an electronic circuit (circuitry) that forms the functional components by reading the program like a CPU. Also, an electronic circuit forming one device may include a plurality of CPUS.

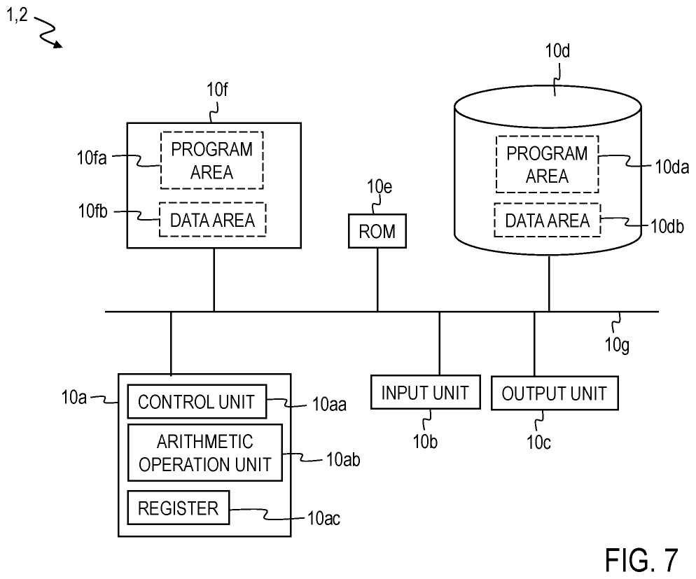

FIG. 7 is a block diagram illustrating an example hardware configuration of the synthetic light source generation device 11 or 11′ and the pupil diameter change induction device 12 in each embodiment. As illustrated in FIG. 7, the synthetic light source generation device 11 or 11′ and the pupil diameter change induction device 12 in this example includes a central processing unit (CPU) 10a, an input unit 10b, an output unit 10c, a random-access memory (RAM) 10d, a read-only memory (ROM) 10e, an auxiliary storage device 10f, and a bus 10g. The CPU 10a in this example includes a control unit 10aa, an arithmetic operation unit 10ab, and a register 10ac, and performs various arithmetic operations in accordance with various programs read into the register 10ac. The input unit 10b is an input terminal to which data is input, a keyboard, a mouse, a touch panel, or the like. The output unit 10c is an output terminal from which data is output, a display, or a LAN card or the like controlled by the CPU 10a that has read a predetermined program. The RAM 10d is a static random-access memory (SRAM), a dynamic random-access memory (DRAM), or the like, and incudes a program area 10da in which a predetermined program is stored and a data area 10db in which various kinds of data are stored. The auxiliary storage device 10f is a hard disk, a magneto-optical disc (MO), a semiconductor memory, or the like, for example, and includes a program area 10fa in which a predetermined program is stored and a data area 10fb in which various kinds of data are stored. The bus 10g connects the CPU 10a, the input unit 10b, the output unit 10c, the RAM 10d, the ROM 10e, and the auxiliary storage device 10f so that information can be exchanged among these components. The CPU 10a writes, into the program area 10da of the RAM 10d, the program stored in the program area 10fa of the auxiliary storage device 10f in accordance with a read operating system (OS) program. Likewise, the CPU 10a writes, into the data area 10db of the RAM 10d, the various kinds of data stored in the data area 10fb of the auxiliary storage device 10f. Addresses in the RAM 10d at which the program and the data have been written are stored into the register 10ac of the CPU 10a. The control unit 10aa of the CPU 10a sequentially reads these addresses stored in the register 10ac, reads the program and the data from the areas in the RAM 10d indicated by the read addresses, causes the arithmetic operation unit 10ab to sequentially execute arithmetic operations indicated by the program, and stores results of the arithmetic operations into the register 10ac. With such a configuration, the functional configurations of the synthetic light source generation device 11 or 11′ and the pupil diameter change induction device 12 are achieved.

The program mentioned above can be recorded in a computer-readable recording medium. Examples of the computer-readable recording medium include a non-transitory recording medium. Examples of such a recording medium include a magnetic recording device, an optical disc, a magneto-optical recording medium, and a semiconductor memory.

The program is distributed by selling, giving, or renting portable recording media such as DVDs or CD-ROMs recording the program thereon, for example. Furthermore, a configuration in which the program is stored in a storage device in a server computer and is distributed by transfer from the server computer to other computers via a network may also be adopted. As described above, the computer executing such a program first stores a program recorded in a portable recording medium or a program transferred from the server computer temporarily into a storage device of the computer, for example. At the time of execution of a process, the computer reads the program stored in the storage device of the computer, and performs processing in accordance with the read program. Also, in other modes of execution of the program, the computer may read the program directly from a portable recording medium and performs processing in accordance with the program, or alternatively, the computer may sequentially perform processing in accordance with a received program every time a program is transferred from the server computer to the computer. Also, the above processing may be performed by a so-called application service provider (ASP) service that implements a processing function only through an execution instruction and result acquisition, without transferring the program from the server computer to the computer. Note that the program in this mode includes information that is to be used in the process by an electronic computer, and is equivalent to the program (data and the like that are not direct commands to the computer but have properties that define the processing to be performed by the computer).

Although this device is formed with a computer executing a predetermined program in each embodiment, at least some of the processing contents may be realized by hardware.

Note that the present invention is not limited to the embodiments described above. For example, the above various kinds of processes may be performed not only in time series in accordance with the description, but may also be performed in parallel or individually depending on the processing ability of the device that performs the processes or as necessary. In addition to the above, it is needless to say that appropriate modifications can be made without departing from the scope of the present invention.

### REFERENCE SIGNS LIST

1 pupil diameter change inducing system
11, 11′ synthetic light source generation device
12 pupil diameter change induction device
113, 113′ weight coefficient generation unit
114 output signal generation unit
123 signal output unit

1. A synthetic light source generation device comprising:
a weight coefficient generation processing circuitry that generates at least a first weight coefficient combination and a second weight coefficient combination corresponding to spectral distributions of a plurality of basic source lights, using a unit LMS chromaticity space value that is a value of an LMS chromaticity space of each basic source light of the plurality of basic source lights having different spectral distributions and a basic ipRGC activation amount that is an ipRGC activation amount for each basic source light of the plurality of basic source lights; and
an output signal generation processing circuitry that generates an output signal, wherein
a rate of change between an ipRGC activation amount of a first synthetic source light having a synthetic spectral distribution obtained by weighting and adding the spectral distributions of the plurality of basic source lights with the first weight coefficient combination and an ipRGC activation amount of a second synthetic source light having a synthetic spectral distribution obtained by weighting and adding the spectral distributions of the plurality of basic source lights with the second weight coefficient combination is higher than a rate of change between a value of an LMS chromaticity space of the first synthetic source light and a value of an LMS chromaticity space of the second synthetic source light,
the output signal includes:
first information for adjusting an output intensity of each basic light source of a plurality of basic light sources that emit the plurality of basic source lights in accordance with the first weight coefficient combination and presenting the first synthetic source light with the plurality of basic light sources in a first time section; and
second information for adjusting an output intensity of each basic light source of the plurality of basic light sources in accordance with the second weight coefficient combination and presenting the second synthetic source light with the plurality of basic light sources in a second time section different from the first time section, and
the output signal is information for repeating presentation of the first synthetic source light and presentation of the second synthetic source light to induce a change in pupil diameter of a presentation target.

2. The synthetic light source generation device according to claim 1, wherein
the weight coefficient generation processing circuitry generates at least the first weight coefficient combination and the second weight coefficient combination, using a target LMS chromaticity space value that is a value of an LMS chromaticity space of a target source light, a target ipRGC activation amount that is an ipRGC activation amount set as appropriate for the target source light, the unit LMS chromaticity space value, and the basic ipRGC activation amount,
N and I are integers of 2 or greater, n=1, . . . , N, and i=1, . . . , I,
L, M, and S represent the target LMS chromaticity space values, and L_{LEDn}, M_{LEDn}, and S_{LEDn} represent the unit LMS chromaticity space values of the nth basic source light of the plurality of basic source lights,
ipRGC_{LEDn} represents an ipRGC activation amount of the nth basic source light,
ipRGC(i) represents the ith target ipRGC activation amount,
w(i) represents the ith weight coefficient combination w₁(i), . . . , and w_{N}(i),
at least some elements of w(i₁) or a weight coefficient combination close to w(i₁) represent the first weight coefficient combination w_{φ(1, 1)}(i₁), . . . , and w_{φ(1, N1)}(i₁),
at least some elements of w(i₂) or a weight coefficient combination close to w(i₂) represent the second weight coefficient combination w_{φ(2, 1)}(i₂), . . . , and w_{φ(2, N2)}(i₂),
i₁, i₂∈{1, . . . , I},
i₁≠i₂,
N1 and N2 are positive integers equal to or smaller than N,
{φ(1,1), . . . , φ(1, N1)}⊆{1, . . . , N},
{φ(2, 1), . . . , φ(2, N2)}⊆{1, . . . , N},
Yw(i)=x(i),
the following expressions are satisfied:
$$Y = ( \begin{matrix}{L}_{{LED}_{1}} & … & {L}_{{LED}_{N}} \\ {M}_{{LED}_{1}} & … & {M}_{{LED}_{N}} \\ {S}_{{LED}_{1}} & … & {S}_{{LED}_{N}} \\ {ipRGC}_{{LED}_{1}} & … & {ipRGC}_{{LED}_{N}}\end{matrix} )$$
$$w ( i ) = ( \begin{matrix}{w}_{1} ( i ) \\ ⋮ \\ {w}_{N} ( i )\end{matrix} )$$
$$x ( i ) = ( \begin{matrix}L \\ M \\ S \\ ipRGC ( i )\end{matrix} )$$
λ represents wavelength,
Iₛ₍ᵢ₁₎(λ)=w_{φ(1, 1)}(i₁)*LED_{φ(1, 1)}(λ)+ . . . +w_{φ(1, N1)}(i₁)*LED_{φ(1, N1)}(λ) represents a synthetic spectral distribution obtained by weighting and adding spectral distributions LED_{φ(1, 1)}(λ), . . . , and LED_{φ(1, N1)}(λ) of the plurality of basic source lights with the first weigh coefficient combination w_{φ(1, 1)}(i₁), . . . , and w_{φ(1, N1)}(i₁), and
Iₛ₍ᵢ₂₎(λ)=w_{φ(2, 1)}(i₂)*LED_{φ(2, 1)}(λ)+ . . . +w_{φ(2, N2)}(i₂)*LED_{φ(2, N2)}(λ) represents a synthetic spectral distribution obtained by weighting and adding spectral distributions LED_{φ(2, 1)}(λ), . . . , and LED_{φ(2, N2)}(λ) of the plurality of basic source lights with the second weight coefficient combination w_{φ(2, 1)}(i₂), . . . , and w_{φ(2, N2)}(i₂).

3-4. (canceled)

5. A pupil diameter change induction device comprising
a pupil diameter change induction signal output processing circuitry that outputs an output signal, the output signal including first information for adjusting an output intensity of each basic light source of a plurality of basic light sources having different spectral distributions and presenting a first synthetic source light with the plurality of basic light sources in a first time section, and second information for adjusting an output intensity of each basic light source of the plurality of basic light sources and presenting a second synthetic source light with the plurality of basic light sources in a second time section different from the first time section, wherein
a rate of change between an ipRGC activation amount of the first synthetic source light and an ipRGC activation amount of the second synthetic source light is higher than a rate of change between a value of an LMS chromaticity space of the first synthetic source light and a value of an LMS chromaticity space of the second synthetic source light, and
the output signal is information for repeating presentation of the first synthetic source light and presentation of the second synthetic source light to induce a change in pupil diameter of a presentation target.

6. (canceled)

7. A synthetic light source generation method comprising:
a weight coefficient generation step of generating at least a first weight coefficient combination and a second weight coefficient combination corresponding to spectral distributions of a plurality of basic source lights, using a unit LMS chromaticity space value that is a value of an LMS chromaticity space of each basic source light of the plurality of basic source lights having different spectral distributions and a basic ipRGC activation amount that is an ipRGC activation amount for each basic source light of the plurality of basic source lights; and
an output signal generation step of generating an output signal, wherein
a rate of change between an ipRGC activation amount of a first synthetic source light having a synthetic spectral distribution obtained by weighting and adding the spectral distributions of the plurality of basic source lights with the first weight coefficient combination and an ipRGC activation amount of a second synthetic source light having a synthetic spectral distribution obtained by weighting and adding the spectral distributions of the plurality of basic source lights with the second weight coefficient combination is higher than a rate of change between a value of an LMS chromaticity space of the first synthetic source light and a value of an LMS chromaticity space of the second synthetic source light,
the output signal includes:
first information for adjusting an output intensity of each basic light source of a plurality of basic light sources that emit the plurality of basic source lights in accordance with the first weight coefficient combination and presenting the first synthetic source light with the plurality of basic light sources in a first time section; and
second information for adjusting an output intensity of each basic light source of the plurality of basic light sources in accordance with the second weight coefficient combination and presenting the second synthetic source light with the plurality of basic light sources in a second time section different from the first time section, and
the output signal is information for repeating presentation of the first synthetic source light and presentation of the second synthetic source light to induce a change in pupil diameter of a presentation target.

8. A pupil diameter change induction method comprising
a pupil diameter change induction signal output step of outputting an output signal, the output signal including first information for adjusting an output intensity of each basic light source of a plurality of basic light sources having different spectral distributions and presenting a first synthetic source light with the plurality of basic light sources in a first time section, and second information for adjusting an output intensity of each basic light source of the plurality of basic light sources and presenting a second synthetic source light with the plurality of basic light sources in a second time section different from the first time section, wherein
a rate of change between an ipRGC activation amount of the first synthetic source light and an ipRGC activation amount of the second synthetic source light is higher than a rate of change between a value of an LMS chromaticity space of the first synthetic source light and a value of an LMS chromaticity space of the second synthetic source light, and
the output signal is information for repeating presentation of the first synthetic source light and presentation of the second synthetic source light to induce a change in pupil diameter of a presentation target.

9. A non-transitory computer-readable recording medium storing a program for causing a computer to function as the synthetic light source generation device according to claim 1.

10. A non-transitory computer-readable recording medium storing a program for causing a computer to function as the pupil diameter change induction device according to claim 5.
